(19) United States
(12) Patent Application Publication
(10) Pub. No.: US 2003/0206614 A1
(43) Pub. Date: Nov. 6, 2003

(54) Method and apparatus for alignment of medical radiation beams using a body frame

(76) Inventors: Lance A. Kendrick, Lafayette, CO (US); Sanford L. Meeks, Iowa City, IA (US)

Correspondence Address: Brian J Keating, Stallman & Pollock, Suite 290, 121 Spear Street, San Francisco, CA, 94105, US

(21) Appl. No.: 10/181,674

(22) PCT Filed: Jan. 30, 2001
(86) PCT No.: PCT/US01/03042

(30) Foreign Application Priority Data
Jan. 31, 2000 (US) 60179043

(51) Int. Cl. A61B 6/08

(52) U.S. Cl. 378/205

(57) ABSTRACT

An apparatus and method of irradiating a patient with radiation beams that converge at an isocenter. The patient is constrained to a frame which is helpful in substantially aligning the isocenter with a target tissue, such as a tumor, in the patient.

### FIELD OF INVENTION

The present invention is directed generally to image-guided medical procedures, and more particularly, to radiation therapy systems requiring high accuracy localization and placement of a patient undergoing a radiation treatment.

### BACKGROUND OF THE INVENTION

Radiation therapy has been used for years in the treatment of tumors. Early methods of treatment used large fields of radiation to destroy tumor cells, but had the adverse effect of harming healthy tissue surrounding the tumor. Advances in this field include conformal radiation therapy, where a target is irradiated from multiple angles by a radiation source which is rotated around the patient. Applying the radiation in this fashion concentrates the energy onto the target tumor while diffusing (or diluting or minimizing) the exposure over healthy tissue, thus minimizing its damaging effects. Radiation therapy machines generate a high energy beam of radiation that is focused on a point called the isocenter over all angles of rotation of the beam. Accurate placement of the tumor at the isocenter location is critical for effective treatment. As accuracy improves, narrower beams of energy can be used with greater precision, thus reducing the damage to healthy tissue.

For the treatment of intracranial tumors, the relatively new technique of stereotactic radiosurgery has been able to provide positional beam accuracies to within a fraction of a millimeter. In this process, a stereotactic frame is rigidly attached to the patient by placing fasteners directly into the skull; afterwards, a detailed three-dimensional image map is produced. Such images can be created from Computer Tomography (CT), Magnetic Resonance Imaging (MRI), or from a variety of other sources. Fiducial markers, placed within or upon the stereotactic frame, appear in the images as reference points. These fiducial markers and allow objects seen in the image to be related to the stereotactic frame, and thus to be related to the patient's anatomy. Once the tumor has been identified in the pre-procedural images, its location can be described using the coordinates of the stereotactic frame. The patient is then transferred to a therapy machine for radiation treatment. The stereotactic frame must be carefully aligned so that area identified as target tissue in the images is placed at the isocenter of the therapy machine. In order to aid in this alignment process, the stereotactic frame may contain trackable markers, such as for example, emitters or reflectors, which can be used to localize the frame relative to some external reference space. Once the positions of the isocenter and the frame are accurately known in the external reference space, the alignment can be readily performed.

Systems have been developed in recent years, which provide accurate position information to assist physicians during medical procedures. Such systems are disclosed, for example, in U.S. Pat. No. 5,588,430 to Bova et al. and PCT Application No. PCT/US95/12894 (Publication No. WO 96/11624) to Bucholz, the entire disclosures of which are incorporated by reference.

For procedures involving the treatment to tumors inside the body, reasonably accurate placement of a tumor, surrounded by soft tissue, at the isocenter position is conducive to a successful treatment. Bony structures can provide a place to which a stereotactic frame may be rigidly attached so that the patient alignment can readily be performed. Prior art methods of body alignment employ the use of lasers and skin markers to perform the alignment. Methods of fixating the patient can include direct body contact which can stretch the skin and alter the position of the skin markers. Prior to treatment, these reference points are applied by the medical technician directly to the patient's body using felt tip markers or reflective adhesive tape. A number of laser beams are aimed at the patient from orthogonal directions to intersect these reference points. The patient is maneuvered by the medical technician so the projected beams fall onto all the marker reference points in order to correctly align the patient for radiation treatment. Another prior art method of bodyframe alignment utilizes scales directly attached to the bodyframe which provide reference marks for the laser beams.

### SUMMARY OF THE INVENTION

The inventors of the present invention have recognized some drawbacks or shortcomings in the prior art. One drawback of prior art methods is in accuracy of targeting the tumor location (relative to the location of the radiation beams). Skin being elastic can move relative to the body's interior, and the resulting movement may introduce errors into position determination. Laser beams can also spread as they propagate through air which may widen the area of the intersection projected on the reference marker and introduces an uncertainty. Reducing the amount of time it takes to achieve patient alignment is also desirable. Furthermore, monitoring orientation of the patient in real time is beneficial. The patient may fall out of alignment during the treatment. Finally, lasers can restrict the orientations of the patient to those which are perpendicular to the coordinate system of the therapy machine. In light of the foregoing, there is a need for an improved devise and method for the efficient and accurate alignment of a patient to a radiation therapy machine.

The present invention is directed generally to stereotactic medical procedures, and, particularly to radiation therapy systems and methods requiring high accuracy localization and placement of a patient undergoing radiation treatment. More specifically, an object of the present invention is directed to a real-time apparatus and method for precisely locating a bodyframe containing a patient to the isocenter of a linear accelerator radiation therapy machine.

To achieve these objects and other advantages and in accordance with the purpose of the invention, as embodied and broadly described herein, the invention is directed to an image guided system for use in performing patient alignment for radiation treatment procedures.

In one aspect of the present invention, a bodyframe containing a plurality of markers is firmly attached to the body of a patient. The markers are imaged by an external electronic detector array which is coupled to a computer. The detector data is processed so that the bodyframe can be accurately localized with respect to the external detector. Furthermore, the computer also receives pre-procedural CT/MRI image data which localizes target tissue with respect to the bodyframe. By combining the detector data (which localizes target tissue with respect to the bodyframe) and the pre-procedural image data, the computer determines the location of the target tumor. By comparing this location with a previously-determined isocenter location, the computer can generate the magnitude and direction of the adjustments required to align the tumor with the isocenter. These adjustments are then displayed for the technician to use to manually align the patient. Furthermore, these values are continuously computed and displayed in real time, providing the technician with the continuous indication of patient alignment throughout the duration of the procedure.

In another aspect of the present invention, the displacements as computed in the foregoing description are fed back into the positioning apparatus so that alignment of the patient can be performed automatically without human intervention.

The present invention overcomes the problems of the prior art by improving the positioning accuracy currently used by laser alignment systems. The prior art approaches have accuracies around 5-10 mm; while at least some embodiments of the invention have accuracies that are at least an order of magnitude better. At least some embodiments of the present invention also significantly improve the ease of positioning the bodyframe by providing direct and continuous indication of the position to the therapist. Furthermore, at least some embodiments of the present invention enable real-time positioning information to be provided throughout the patient setup and radiation delivery phases of the treatment. Finally, the ability to utilize non-orthogonal positions of the bodyframe relative to the therapy machine coordinate frame is provided by at least some embodiments of the present invention.

It is to be understood that both the foregoing general description and the following detailed description are exemplary and explanatory only and are not restrictive of the invention.

Further applicability of the present invention will become apparent from a review of the detailed description and accompanying drawings. It should be understood that the description and examples, while indicating preferred embodiments of the present invention, are not intended to limit the scope of the invention, and various changes and modifications within the spirit and scope of the invention will become apparent to those skilled in the art.

### BRIEF DESCRIPTION OF THE DRAWINGS

The accompanying drawings, which are incorporated in and constitute a part of this specification, illustrate several embodiments of the present invention and together with the description, serve to explain the principles of the invention.

FIG. 1 is a simplified side view of a preferred embodiment of the present invention showing a patient undergoing treatment with beams of radiation.

FIG. 2a is a top view of the bodyframe showing the tracking markers.

FIG. 2b is a side view of the bodyframe showing the tracking markers.

FIG. 3 is a block diagram of the computer system which is used in the present invention.

FIG. 4 is a diagram of the display showing the manner for indicating adjustments for patient positioning.

FIG. 5 is a flowchart showing the steps executed under a preferred embodiment of the present invention to perform alignment.

FIG. 6 shows a simplified view of an alternate embodiment of the present invention.

### DETAILED DESCRIPTION OF THE PREFERRED EMBODIMENTS

Before starting a description of the Figures, some terms will now be defined.

Definitions

the present invention or the invention: means at least some embodiments of the present invention; references to various feature(s) of the “present invention” throughout this document do not mean that all claimed embodiments or methods include the referenced feature(s).

“location of convergence of the radiation beams” and “displacing the radiation beams:” according to preferred embodiments of the present invention, the radiation beams are not turned on during the time that their proposed location, with respect to patient and frame, is initially determined and then adjusted or aligned. Rather, the beams are preferably only activated after the alignment is complete. Accordingly, when the claims refer to the location of radiation beams or displacement of the isocenter, this reference refers to either the actual location of the radiation beams and/or isocenter that are turned on or the location that the radiation beams and/or isocenter would occupy if they were turned on. Notwithstanding this broad definition of radiation beams, the claims are intended to encompass embodiments of the present invention wherein: (1) the radiation beams are turned on during the alignment; (2) the radiation beams are turned off during the alignment; and (3) the radiation beams are set at some intermediate power setting between on and off during the alignment.

Reference will now be made in detail to the present preferred embodiments of the invention, examples of which are illustrated in the accompanying drawings. Whenever possible, the same reference numbers will be used throughout the drawings to refer to the same or like parts.

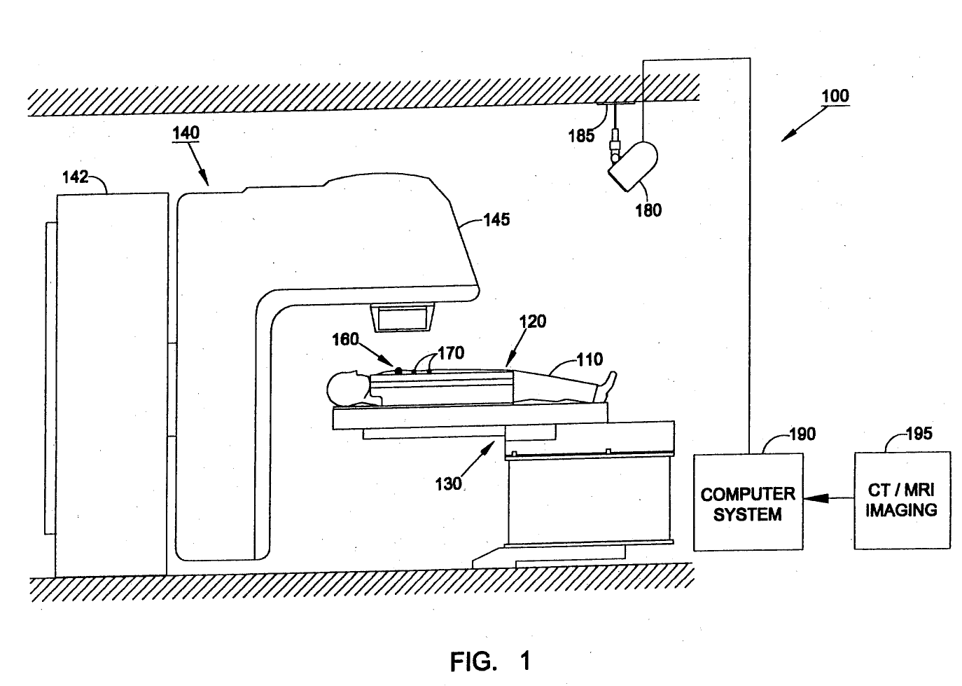

FIG. 1 illustrates the system 100 for implementing the present invention. Patient 110 is firmly affixed to a bodyframe 120 which is supported by a moveable platform 130. The patient lies under a therapy machine 140, for example, a linear accelerator may be used which emits X-ray or electron beams. The gantry 145 of machine 140 rotates around the longitudinal axis of patient 110 and directs a narrow beam of radiation towards the isocenter 160. The radiation therapy machines of this type are well known in the art.

Patient 110 is positioned by moveable platform 130 to accurately place the portion of the patient's body containing a tumor at the isocenter position. Platform 130 can be moved in the horizontal (both laterally and longitudinally) and vertical positions, and can also be rotated about its vertical axes of rotation. Movable platforms of this type are well known in the art.

Bodyframe 120 is used to immobilize the patient, and it may be rigidly mounted to the moveable platform 130. Bodyframe 120 includes a number of tracking markers 170 that are detected or sensed by a sensor, such as an electronic detector array 180. Suitable tracking markers known in the art, such as for example markers 170, also serve as a reference position frame needed to establish coordinates for the patient's body. The detector array is suspended by mount 185 and located in such a manner as to provide detector array 180 with a clear line of sight to the tracking markers 170.

The manner in which the detector tracks the markers, such as for example detector array 180 tracks the positions of the bodyframe, is well known in the art and is therefore only described generally. The array 180 includes a plurality of detectors for tracking positions. The detectors can utilize CCD devices to detect the tracking markers 170. Based on the relative coordinates of these detected markers 170, the positions of the objects can be determined, or localized, within the frame of reference of the detector array. For purposes of this description, this external reference is defined as the detector space.

The detector array 180 is coupled to a computer system 190 programmed with software modules that analyze the signals transmitted by the detector array to determine the position of the bodyframe in the detector space. Computer 190 also receives a pre-procedural image data set 195 of the body site of interest usually generated by some scanning technique such as CT imaging or MRI. These images are obtained prior to the radiation procedure and are used to precisely locate the target tissue inside the patients body, as will be described more fully below. Through the appropriate combination of pre-procedural image data and detector array data, the system can localize the tumor in detector space. Once localized, its position can be compared with the previously calibrated position of the therapy machine isocenter and the displacements between the two points are displayed on a monitor connected to computer 190 (not shown). This process is computed and displaced on the monitor in real-time during the procedure. The tracking technology employed in the present invention may be the same as that used in the STEALTH STATION or Radio Cameras system available from Medtronic Sofamor Danek, Inc. (It is noted that the terms “STEALTH STATION,” “Radio Cameras” and/or “Medtronic Sofamor Danek” may be subject to trademark rights.)

Prior to undergoing radiation therapy, the patient is placed in the bodyframe 120 and a set of three-dimensional images are produced using CT imaging, MRI scans, or some other scanning method. Fiducial markers are located on the bodyframe and appear as reference marks on the image set. The fiducial markers may be separate entities whose location is precisely known relative to the tracking markers 170; or, as in the preferred embodiment, the same physical markers can be used as both fiducial and tracking markers. The points where the fiducial markers are located define the bodyframe coordinate system and, hence, points on the patient's anatomy since the bodyframe is firmly attached to the patient. Features which appear in the pre-procedural CT/MRI image sets can be directly related to the bodyframe by exploiting the reference points, which correspond to the bodyframe fiducial markers, in the image. After the tumor has been identified and localized in the bodyframe coordinate space, the patient is later transferred to the therapy machine for treatment. The bodyframe 120 is placed on moveable platform 130 in view of detector array 180. Tracking markers 170 are sensed by detector 180 and their position is localized by computer 190 in detector space as is known in the prior art. The position of the tumor, defined in bodyframe space, is derived from the pre-procedural image data 195 and combined with the marker position data to localize the tumor in detector space. By computing the displacement between the tumor location and pre-calibrated position of the isocenter, values representing the misalignment between the two can be displayed for the medical technician. The technician then adjusts the movable platform 130 so that the misalignment is minimized or eliminated. Once this occurs, radiation therapy can commence. Throughout the entire procedure, the misalignment data is computed and displayed in real time, so that if the patient becomes misaligned while radiation is being applied, the technician can instantly recognize this and respond by temporarily interrupting the radiation treatment and manually correcting the misalignment. Alternatively, any misalignment can be automatically corrected by a suitably equipped computer 195. Additionally, computer 195 can also be used to automatically disengage the radiation treatment.

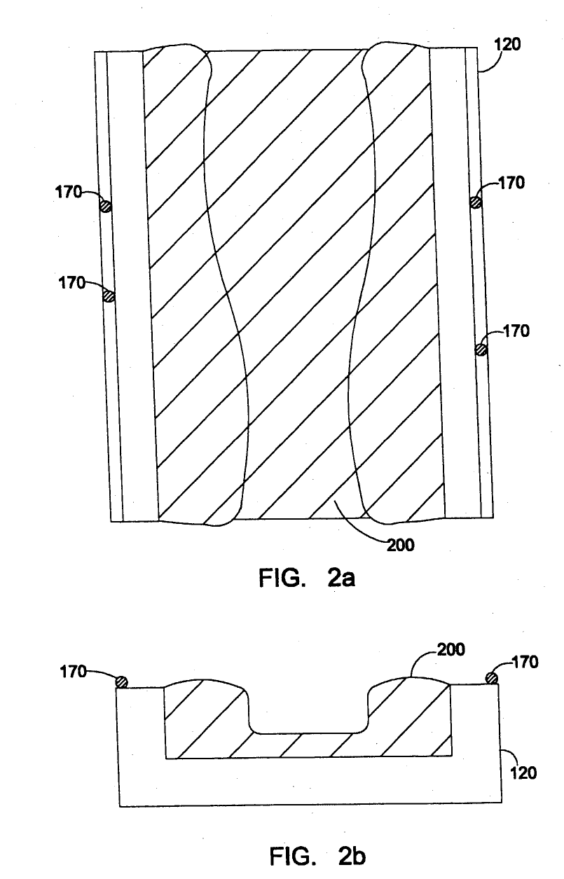

A top view of the bodyframe is shown in FIG. 2a. The frame is made of a material which will not interfere with either scanning operation nor the tracking operation that is to be performed. One material suitable for construction of bodyframe 120 (when MRI scans are to be used) is polycarbonate, or other suitable materials, such as carbon fiber. The patient is firmly attached to the bodyframe through an attachment member 200. Such attachment member can be a custom mold or vacuum pillow which follows the contours of the patient's anatomy, which is well known in the art. For example, bodyframes manufactured by Med-Tec, Medical Intelligence, Midco, and Elekta. (The terms “Med-Tec,” “Medical Intelligence,” “Midco” and/or “Elekta” may be subject to trademark rights.) The bodyframe 120 is equipped with a plurality of tracking markers 170 which are placed in a known geometry. In the preferred embodiment, a plurality of markers 170 (here shown as four, by way of example only) are used for representing the location of the bodyframe in detector space. The markers may be, for example, reflective markers and/or light emitting diodes (LED's). In the preferred embodiment, the markers are spherically shaped reflectors such as those supplied by Northern Digital. (The term “Northern Digital” may be subject to trademark rights.) Other devices known in the art may be used that are capable of being tracked by a corresponding sensor array within the scope of the present invention. For purposes of illustration, and not by limitation, the tracking means may be acoustic, magnetic, optical, electromagnetic, and radiologic devices known in the art or even may take the form of other types of tracking devices to be developed in the future.

FIG. 2b shows a side view of the bodyframe 120 showing attachment member 200 and tracking markers 170.

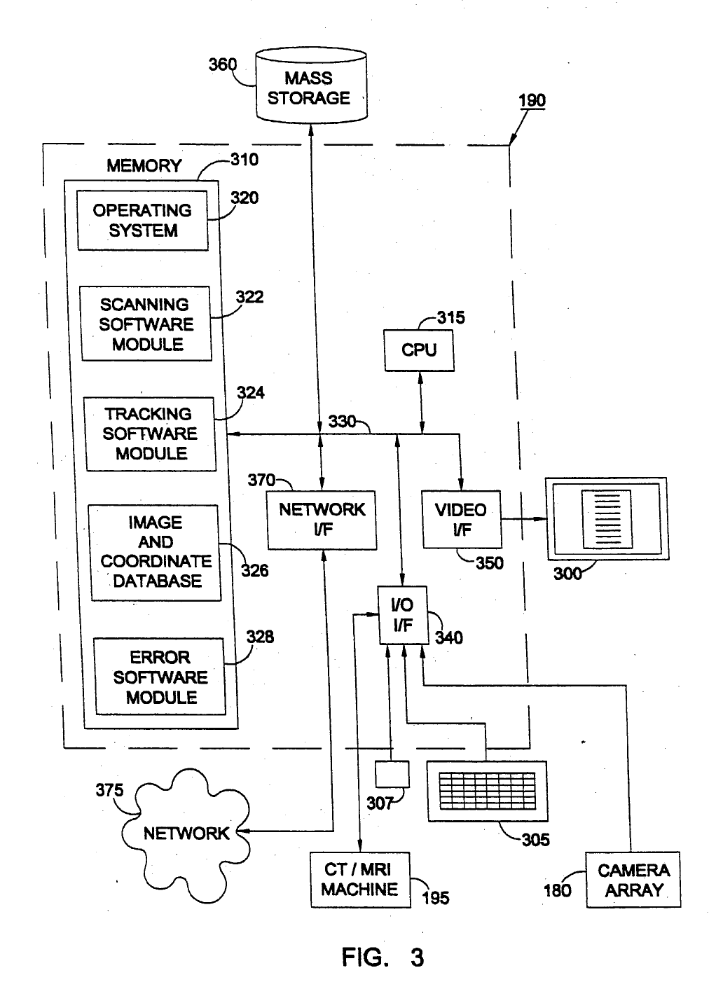

Referring to FIG. 3, the general components and modules of a computer system 190 used to perform various processes of the invention is described. Although a STEALTH STATION image guided system manufactured by Medtronic Sofamor Danek has been identified, it will be appreciated that the present invention may be utilized with other types of computer systems. One embodiment of the computer system 190 includes a graphical user interface system operating in conjunction with a display screen of a display monitor 300. The graphical user interface system is preferably implemented in conjunction with the operating system of computer 190 for displaying and managing the display objects of the system. The graphical user interface is implemented as part of the computer system 190 to receive input and commands from a conventional keyboard 305 and mouse 307. For simplicity of the drawings and explanation, many components of a conventional computer system have not been illustrated such as address buffers, memory buffers, and other standard control circuits because these elements are well known in the art and a detailed description thereof is not necessary for understanding the present invention. A computer program used to implement the various steps of the present invention is generally located in memory unit 310, and the processes of the invention are carried out through the use of a central processing unit (CPU) 315. Those skilled in the art will appreciate that the memory unit 310 is representative of both read-only memory and random access memory or any other type of memory now known or developed in the future. The memory unit also contains a database 326 that stores data, for example, image data and tables, including such parameters as the isocenter location in detector space, used in conjunction with the invention. CPU 315, in combination with the computer software, comprising operating system 320, scanning software module 322, tracking software module 324, and error software module 328 controls the operations and processes of the computer system 190. The process implemented by CPU 315 may be communicated as electrical signals along bus 330 to an I/O interface 340 and a video interface 350. Scanning software module 322 performs the processes associated with creating a coordinate reference system and reference images for use in connection with the present invention and are known to those skilled in the art. Tracking software module 324 performs the processes necessary for tracking objects in an image guided system as described herein and are known to those skilled in the art. Error software module 328 computes the displacements between the target and isocenter and converts this data to values most suitable for operator use. These values are subsequently displayed on monitor 300 through video interface 350. Images from the CT or MRI machine 195 can be fed directly into computer 190 through I/O interface 340, or may be supplied through a removable mass storage device 360. Furthermore, pre-procedural images may also be supplied over a network 375 through a network interface 370.

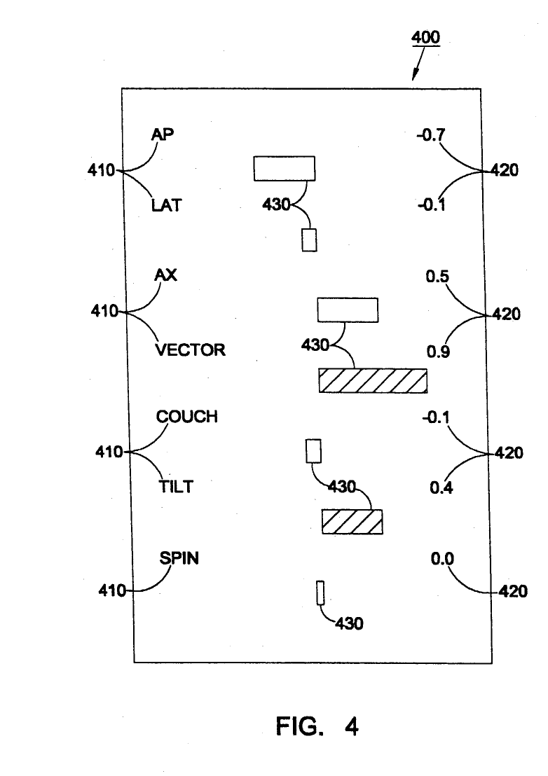

FIG. 4 shows an exemplary diagram of display 400 which may be presented to the medical technician on monitor 300 of computer generated system 190. The display shows identifying labels 410 of various displacement and rotation parameters which may be adjusted to align the patient by issuing commands to the movable platform 130. The displacement parameters are described by the following axes: the anterior-posterior (AP), which lies perpendicular to table 130; the lateral (LAT), which lies in the plane of table 130 parallel to its short edge; and the axial (AX) axis which lies in the plan of table 130 parallel to its long axis. The three rotational parameters are: couch, which rotates about the AP axis; spin, which rotates about the AX axis; and tilt, which rotates about the lateral axis. Numerical values 420 representing the magnitudes and directions of each alignment parameter are displayed alongside to its corresponding label 410. The numerical value represents the displacement from the isocenter. Graphical representations 430 of each parameter are also displayed as bar graphs. The length of each symbol is representative of the magnitude of the representative parameter and its direction is also displayed. Color coding of each symbol can also be used to alert the medical technician of certain parameters which require immediate attention. Obviously, other parameters could be shown on the display.

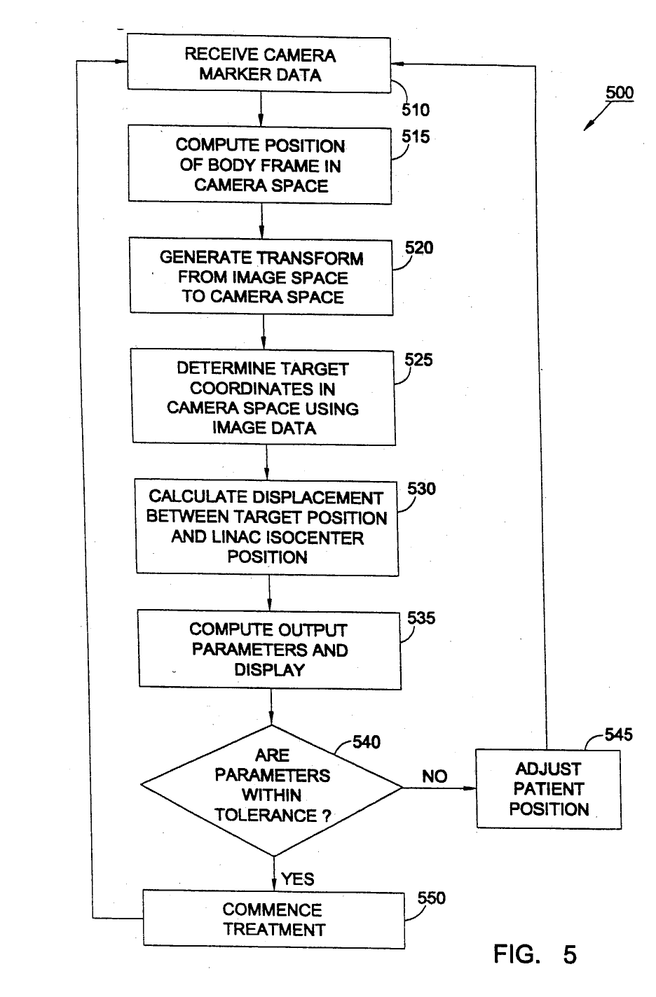

Referring to FIG. 5, the processes or steps associated with alignment procedures is illustrated at 500. These procedures take place while the patient is located on moveable platform 130 of system 100 and after the pre-procedural images of the patient have been generated. Initially, marker data is received by computer 190 from detector array 180 and pre-processed to identify the markers in the detector array output (step 510). Position data for bodyframe 120 is computed from the detector data and described in detector space (step 515). Once the marker positions are known in detector space, a transform from pre-procedural image space to detector space can be computed given the positions of the fiducial markers in the pre-procedural images and image pixel spacing (step 520). The target coordinates of the tumor are then extracted from the pre-procedural images, and utilizing the transform obtained in step 520, the target coordinates of the tumor in detector space are computed (step 525). Given the previously-calibrated position of the isocenter in detector space, the displacement between the tumor position, calculated in step 525, and the isocenter is computed (step 530). The displacement values are then converted to parameters which are readily interpreted by a medical technician and displayed on monitor 300 of computer system 190 in a format shown by 400 in FIG. 4 (step 535). If all the parameters are within predetermined tolerances, the radiation treatment can begin (step 550). If not, the medical technician will adjust the patient position (step 545) to eliminate or minimize any misalignment. In either event, the process returns to step 510 in order to provide continuous monitoring of patient alignment throughout the entire treatment process.

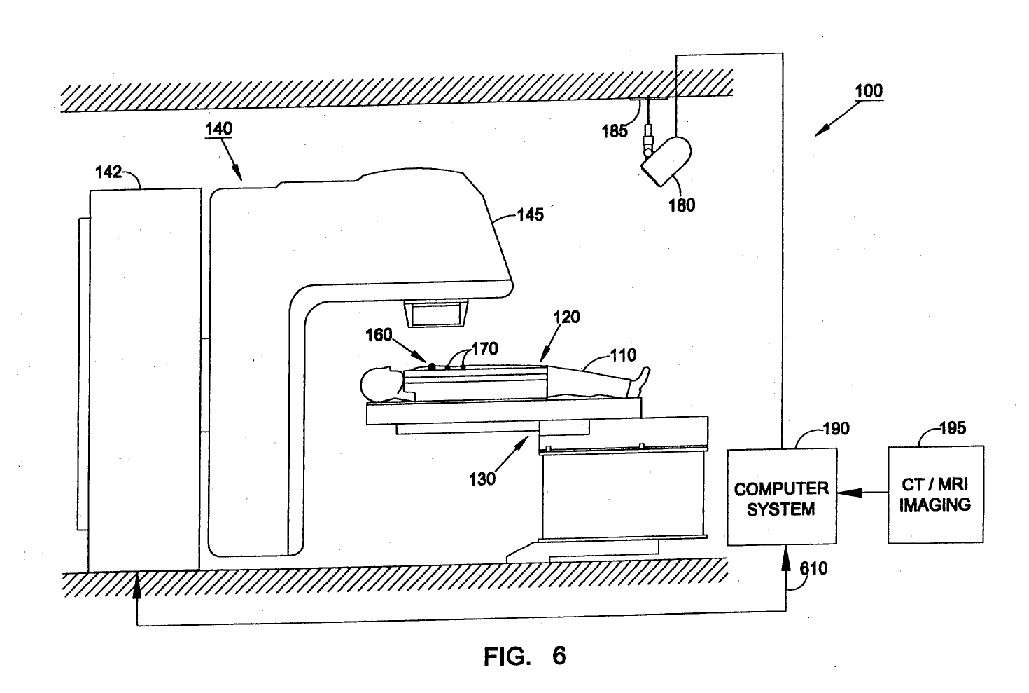

FIG. 6 shows an alternate embodiment of the present invention. This embodiment is identical to that shown in FIG. 1 with the exception that the entire alignment process is automatic. In this embodiment, the medical technician is not required for manually adjusting the patient position in accordance with the values presented on the software display 400. Radiation machine controller 142 contains the necessary components to control gantry 145 and platform 130. An electrical connection 610 between computer system 190 and controller 142, provide the means for the computer system to automatically control the patient alignment based upon the steps shown in FIG. 5. Process 500 will continue as before until step 540 is completed. Computer system 190 determines if the alignment parameters are within tolerance (step 540), and if so, signal the therapist to begin treatment (step 550). On the other hand, if the patient is not aligned at step 540, the computer will signal the radiation machine controller 142 to move the patient in the required directions to eliminate or minimize any misalignment (step 545). After the radiation treatment commences, the computer 190 monitors the alignment process throughout the radiation therapy and makes any adjustments if necessary. Additionally, computer 195 can signal radiation machine 140 to interrupt treatment if required.

What is claimed:

1. A method for positioning a patient (110) for radiation treatment which comprises the steps of:
generating a set of pre-operative images in a region of interest of a patient's body (110) taken while patient occupies a bodyframe (120).
identifying and locating a target issue within the patient's body using the pre-operative images of the body;
tracking the position of the bodyframe with an external tracking system (170, 180);
computing displacements of the target tissue position from an isocenter (160) of a radiation therapy machine; and
aligning the bodyframe according to the output parameters so that the target tissue is substantially coincident with the isocenter.

2. The method of claim 1 further including the step of affixing a region of the patient's body between the patient's neck and the patient's hips in a fixture which substantially conforms to the contours of the patient's body.

3. The method of claim 1 further including the step of generating images using a scanning method.

4. The method of claim 1 further including the step of utilizing at least one fiducial marker on the bodyframe as at least one reference point in the pre-procedural images.

5. The method of claim 1 further including the steps of:
placing a plurality of tracking markers on the bodyframe; and
employing the external tracking system with a computer controller and sensor unit for sensing the tracking markers and tracking the position of the bodyframe in real time.

6. The method of claim 1 further including:
generating a transform from pre-procedural image coordinates to external tracking system coordinates by correlating tracking markers and the fiducial markers; and
utilizing the transform to determine the target tissue coordinates in the external tracking system.

7. The method of claim 1 further including:
continuously tracking the bodyframe; and
computing the displacements and displaying the alignment parameters in real-time.

8. The method of claim 1 further including the step of:
displaying output parameters which represent the displacements.

9. The method of claim 5 further including the steps of:
automatically adjusting positioning to align the bodyframe under control of a computer; and
allowing the computer to disengage the radiation treatment if misalignment occurs during treatment.

10. A method of treating a patient (110) using beams of radiation to irradiate a target tissue, the method comprising the steps of:
constraining the patient to a frame (120);
determining the position of the target tissue with respect to an isocenter (160) defined by a location of convergence of the radiation beams;
aligning the target tissue to be substantially co-incident with the isocenter by displacing at least one of the patient and the isocenter; and
after the alignment step, irradiating the patient with the radiation beams.

11. The method of claim 10, wherein the frame is a bodyframe.

12. The method of claim 10, wherein the patient is constrained by fastening the frame to at least one of the patient's bones.

13. A device for irradiating a target tissue of a patient using beams of radiation that converge at an isocenter (160), the device comprising:
a radiation beam generator (140) structured to generate the radiation beams and to direct the radiation beams to converge at the isocenter;
a frame (120) structured to constrain a patient with respect to the fame;
a computer system (170, 180, 190) structured and programmed to determine the location of the target tissue with respect to the isocenter when the patient is constrained in the frame; and
alignment hardware (130) structured to displace at least one of the patient and the isocenter so that the isocenter is substantially aligned with the target tissue.

14. The device of claim 13 wherein the frame includes fasteners structured to be fastened to bone.
